(19) United States
(12) Patent Application Publication
(10) Pub. No.: US 2003/0148259 A1
(43) Pub. Date: Aug. 7, 2003

(54) Method of identifying insulin secretion stimulating compounds, and the use of such compounds in treating insulin-secretion related disorders

(76) Inventor: Shahidul Islam, Stockholm (SE)

Correspondence Address: LARSON & TAYLOR, PLC, 1199 NORTH FAIRFAX STREET, SUITE 900, ALEXANDRIA, VA, 22314, US

(21) Appl. No.: 10/308,168

(22) Filed: Dec. 3, 2002

Related U.S. Application Data
(60) Provisional application No. 60/351,375, filed on Jan. 28, 2002.

(51) Int. Cl. C12Q 1/00; G01N 33/53; G01N 33/567

(52) U.S. Cl. 435/4; 435/7.2

(57) ABSTRACT

Calcium Induced Calcium Release (CICR) in β-cells has been found to be a novel target for stimulating insulin secretion in a glucose-dependent manner. The present invention relates to a method of identifying compounds that stimulate insulin secretion in a context-dependent manner based on the finding that compounds stimulating insulin secretion in a glucose-dependent manner are able to elicit periodic amplified Ca²⁺ release in β-cells. The invention also relates to the use of such compounds, for the manufacture of a medicament for use in the treatment of defective insulin secretion related disorders, especially type 2 diabetes.

### CROSS-REFERENCE TO RELATED APPLICATIONS

This application claims the benefit of U.S. Provisional Application Ser. No. 60/351,375, filed Jan. 28, 2002

### TECHNICAL FIELD OF THE INVENTION

The present invention relates to a method of identifying compounds that stimulate insulin secretion in a context-dependent manner, and the use of such compounds, or a pharmaceutically acceptable salt thereof, for the manufacture of a medicament for use in the treatment of defective insulin secretion related disorders, especially type 2 diabetes.

### BACKGROUND ART

In the treatment of type 2 diabetes, orally active drugs that stimulate insulin secretion from pancreatic beta cells are extremely important. Commonly used oral hypoglycaemic agents are the sulfonylureas of first, second third generation and the meglitinides. A major problem with the sulfonylurea group of drugs is that they stimulate insulin secretion even when blood glucose concentration is low with the risk of causing fatal hypoglycemia (Herbel, G. & Boyle, P. J. (2000) Endocrinol. Me-tab Clin. North Am. 29, 725-743). It is thus desirable to discover orally active antidiabetic agents that stimulate insulin secretion only when blood glucose concentration is high and does not stimulate secretion when glucose concentration is lowered.

The cellular target of most of the oral hypoglycaemic agents including sulfonylureas and meglitinides is the K_{ATP} channel (Ashcroft, S. J. (2000) J. Membr. Biol. 176, 187-206). Drugs that use K_{ATP} channel as target stimulate insulin secretion irrespective of whether blood glucose concentration is high or low. This is because inhibition of K_{ATP} channel does not depend on prevailing glucose concentration. Moreover anti-diabetic drugs that act on K_{ATP} channel of beta-cells can also act on the cardiovascular K_{ATP} channels increasing the risk for complications (Howes, L. G. (2000) Diabetes Obes. Metab 2, 67-73). Thus it is desirable to identify cellular targets other than K_{ATP} channel, that can be used for stimulating insulin secretion in a glucosedependent manner, and compounds affecting such stimulation.

The present inventor has now found Calcium Induced Calcium Release (CICR) in β-cells to be a novel target for stimulating insulin secretion in a glucosedependent manner, especially CICR involving the ryanodine receptor. More particularly, compounds stimulating insulin secretion in a glucose-dependent manner have surprisingly been found to elicit distinct periodic amplified Ca²⁺ release in β-cells. Accordingly, a method of identifying such compounds based on the above finding is provided by the present invention, which method has been specified in claim 1, including the steps of: A) providing a set of β-cells capable of CICR; B) adding a candidate compound to be tested to the cells; and C) monitoring the cells for periodic amplified Ca²⁺ release in said cells after addition of the candidate compound of step B. In addition, a method of identifying such compounds is also provided which comprises providing a set of β-cells capable of CICR; B) selecting at least one vital/healthy β-cell of said set; C) addition of a candidate compound to be tested to the cell(s) selected in step B; and, monitoring said at least one cell selected in step B for periodic amplified Ca²⁺ release in said cell after addition of the candidate compound of step C.

### SUMMARY OF INVENTION

In a first aspect the present invention relates to a general method of identifying compounds that stimulate insulin secretion in a context-dependent manner, comprising the steps of:

A. providing a set of β-cells capable of CICR;

B. adding a candidate compound to be tested to the cells; and

C. monitoring the cells for periodic amplified Ca²⁺ release in said cells after addition of the candidate compound of step B.

The method can for example be used in high-throughput screening for compounds that stimulate insulin secretion in a context-dependent manner.

In a second aspect the present invention relates to a method of identifying compounds that stimulate insulin secretion in a context-dependent manner, comprising the steps of

A. providing a set of β-cells capable of CICR;

B. selecting at least one viable/healthy β-cell of said set;

C. adding a candidate compound to be tested to the cell(s) selected in step B; and

D. monitoring said cell(s) selected in step B for periodic amplified Ca²⁺ release in said cell(s) after addition of the candidate compound of step C.

By the inclusion in the general method of step B, wherein at least one viable/healthy β-cell of the set provided in step A is selected for monitoring in step D, monitoring will be focused on reliable sources of Ca²⁺ release signals, allowing for more accurate results to be obtained.

By using cells having ryanodine receptors in the methods, greater Ca²⁺ release can be obtained, thereby leading to more easily detected signals. By using an agent reducing the background [Ca²⁺]ᵢ, such as D600 or verapamil, in the method, the CICR component can be visualized better. In the method, a specific type of cell, such as the so called S5 cell, which is particularly adapted to the method, and provides more reproducible CICR, can be used.

The method of detection of [Ca²⁺]ᵢ, and thus CICR, is not critical and can be accomplished by any known method as long as CICR is not inhibited thereby. In any case, care must be taken not to use disturbingly high concentrations of any reagents involved. The method of detection can conveniently be based on fluorescence, using a fluorescent Ca²⁺ indicator.

The methods of the invention offer a reproducible and substantially less time-consuming route of detecting potent compounds stimulating insulin secretion in a glucose-dependent manner, than for example by direct measurement of insulin release.

By means of the methods, the relative potency of an CICR-active agent can be estimated semi-quantitatively from the frequency and amplitude of the amplified Ca²⁺ signals.

In a third aspect the present invention relates to the use of compounds stimulating insulin secretion in a context-dependent manner, identified by means of the method, for the preparation of a pharmaceutical for use in treating diabetes.

Further embodiments and advantages of the invention will be evident form the detailed description hereinafter, and in the appended claims.

### BRIEF DESCRIPTION OF ATTACHED DRAWINGS

FIG. 1A is a control experiment according to Example 1.

In FIG. 1B a test substance (in this case forskolin 5 μM) is also included, which gives rise to the periodic amplification of Ca²⁺ signals, indicating that it is a sensitizer of CICR.

FIG. 2 illustrates a test for screening CICR by identifying periodic amplification of Ca²⁺ signals as described in Example 5. The test substance (in this test glucagons-like peptide 1) gave rise to periodic amplification of Ca²⁺ signals. FIG. 3 shows testing of caffeine and isocaffeine according to Example 6. Caffeine was more potent than isocaffeine and accordingly caffeine gave more frequent CICR. A is the test and B is the control experiment.

FIG. 4 shows similar testing as in FIG. 3, using S5 cells and MBED (50 μM) instead.

FIG. 5A shows the effect of MBED on insulin secretion from a highly differentiated clonal insulin secreting cell line INS-1E. 5B shows the dose response of MBED-induced insulin secretion in INS-1E cells.

FIG. 6 shows stimulation of insulin secretion from islets by caffeine (B) and MBED (D). At times indicated by horizontal bars, the islets were perifused with 11.2 mM glucose with or without sensitizers of RY receptors i.e. 2.5 mM caffeine (B) or 6 μM MBED (D).

FIG. 7 shows confocal images of changes in [Ca²⁺] in INS-1E cells loaded with fluo-3 induced by MBED.

FIG. 8 illustrates effects of caffeine and its analogs on insulin secretion (8A-C) and cAMP-PDEs (8D).

FIG. 9 illustrates the concentration-response relationship showing the extent of inhibition of cAMP-PDEs and of stimulation of insulin secretion by caffeine.

FIG. 10 shows confocal images of changes in [Ca²⁺] in INS-1E cells loaded with fluo-3 induced by caffeine (5 mM) (upper panel), and forskolin (lower panel).

FIG. 11A shows the effects of caffeine (0.75 mM) on insulin secretion in control- and thapsigargin-treated cells in the presence of 3 and 11 mM glucose, and 11B shows the abolishment of glucose-dependent stimulation of insulin secretion by caffeine in the of dantrolene.

### DETAILED DESCRIPTION OF THE PRESENT INVENTION

The present inventor hereby suggests calcium-induced calcium release (CICR) as a novel target for stimulating context-dependent insulin secretion from β-cells. CICR in β-cells was described for the first time in 1992 by Islam, M. S., et al. in FEBS Lett. 296 (3):287-291 (1992), and confirmed by subsequent publications, especially by Islam, M. S., et al. in Proc.Natl.Acad.Sci.U.S.A 95 (11):6145-6150 (1998). Due to the distinctive properties of β-cell ryanodine receptors, the present inventor suggests that drugs acting on this target might be able to stimulate insulin secretion only when glucose concentration is relatively high, i.e., in a context dependent manner. Compounds acting as insulin-secretagogues that stimulate insulin secretion selectively in the presence of high concentration of glucose have been identified by the present inventor. Of the compounds found, 9-methyl-7-bromoeudistomin D hydrochloride is presently believed to be the most potent one.

Generally, according to the findings of the present inventor, compounds that can be used in for context dependent stimulation of insulin secretion according to the present invention are any compounds having an activating effect on the ryanodine receptor, and enhancing effect on the calcium-induced calcium release, and also a stimulatory effect on the Ca²⁺ releasing activity in β-cells. Accordingly, an example of such compounds is caffeine. However, as will be shown in more detail below, the concentration required in order to achieve the desired context dependent release of insulin according to the present invention, in the case of caffeine, is too high for caffeine to be of practical value in the treatment of diabetes. Accordingly, it is preferred that the compounds of the present invention exhibit an activating effect on the ryanodine receptor, enhancing effect on CICR, and also a stimulatory effect on the Ca²⁺ releasing activity already at very low concentration.

Based on the above findings the present inventor has devised a method for identifying compounds stimulating context dependent insulin secretion in β-cells.

CICR is a multi-step process. The molecules and structures that participate in CICR include: 1. the plasma membrane Ca²⁺ channels; 2. intracellular Ca²⁺ release channels; 3. intracellular Ca²⁺ stores; and, 4. a large number of molecules associated with the plasma membrane and the intracellular Ca²⁺ release channels. Regulation of CICR is tissue dependent. For example, in β-cells CICR is a strictly context-dependent process. There is currently no suitable method for screening drugs that act on CICR. The conventional methods for studying CICR in muscle cells use ryanodine-binding assay using microsomes. These methods are indirect and use destructive biochemistry and do not actually study CICR. These methods are not suitable for β-cells also for following reasons: 1. For preparation of microsomes large amounts of pure β-cells are required which are not readily available; 2. the density of ryanodine receptors and other intracellular Ca²⁺ channels in β-cells is low; 3. the coupling between multiple molecules that mediate CICR is lost in microsome preparations; and, 4. metabolism of nutrients is essential for glucose-stimulation of β-cells but such metabolism is absent in microsomes.

For screening drugs that act on CICR in β-cells a method is needed, which can elicit true CICR, which is highly reproducible, which does not require large amount of β-cells and which employs intact and living β-cells. Such a method has now been developed wherein highly reproducible CICR in single living β-cells can be elicited. In this system the interacting molecules that perform CICR are kept intact and the intracellular environment including metabolic potential of β-cells are kept undisturbed. Moreover, the signal to noise ratio in the method is very high, thus allowing confident detection of CICR-active agents. This method is thus suitable for screening large amount of CICR active agents in β-cells.

In order for the β-cells to be able to elicit CICR, the cells require a high glucose concentration, such as for example 10 to 15 mM, typically 10 to 13 mM, and the cells must also be depolarized.

The inventive method will now be disclosed in closer detail.

Screening for CICR-active Agents in β-cells by Detection of Periodic Amplification of Ca²⁺ signals in β-cell.

Since β-cells are difficult to obtain in large numbers we developed a method where single β-cells can be used for screening CICR-active agents in these cells. In this system, whether or not a compound or drug acts by targeting CICR, is determined by detection of periodic amplification of Ca²⁺ signals in β-cells. We have established that these periodic amplifications of Ca²⁺ signals in β-cells are signatures of CICR in β-cells. The method is suitable for screening CICR-active agents irrespective of whether CICR is mediated by the inositol- 1,4,5-trisphosphate receptor (IP3R) or the ryanodine receptor (RyR). It is a method for screening compounds that affect CICR by acting on molecules interacting with the intracellular Ca²⁺ channels. These molecules include calsequestrin, protein kinase A, protein phosphatases, calmodulin, Ca²⁺ calmoldulin dependent protein kinase, ankyrin, FK506-binding protein, calcineurin, and triadin. It is also a method for screening compounds that affect CICR by acting on molecules or signalling pathways that affect intracellular Ca²⁺ channels. These include CD38, BST- 1, cAMP-signalling pathway, nitric oxide signalling pathway. It is also a method for screening drugs that are likely to stimulate insulin secretion only in the presence of a high concentration of glucose, i.e. in a context-dependent manner.

The present method offers a much faster and simplified route for detection of molecules potentially useful in treatment of defective insulin secretion disorders, as compared to the direct monitoring and detection of insulin release. The detection is on-line, and any response is obtained within a few seconds to a few minutes, typically less than 5 minutes.

A potentially useful molecule found by means of this screening method can subsequently be tested for context-dependent stimulation of insulin secretion in β-cells, by adding said molecule to the β-cells and monitoring insulin secretion. This test is not critical and be performed by any suitable method known in the art.

According to the method of the present invention, compounds can be screened for their ability to activate CICR by testing their ability to elicit periodic amplification of Ca²⁺ signals in β-cells. Specific examples of reproducible methods and protocols whereby CICR can be elicited and quantified in β-cells will be described hereinafter.

Periodic amplification of Ca²⁺ signals are large and transient increases in intracellular free Ca²⁺ concentration ([Ca²⁺]ᵢ) that are superimposed on modestly elevated ambient [Ca²⁺]ᵢ. The amplified Ca²⁺ signals occur periodically with intervals ranging from a few seconds to a few minutes. Periodic amplification of Ca²⁺ signals can continue for several minutes or as long as 20-30 minutes. CICR occurs characteristically when ambient [Ca²⁺]ᵢ is slightly elevated, e.g. 200-300 nM. But less occasionally it can occur when basal [Ca²⁺]ᵢ is 50-200 nM. A single amplified Ca²⁺, i.e. a single spike, can also be due to CICR but can be a non-specific incidental finding and is consequently not considered diagnostic of CICR. Periodic amplification of Ca²⁺ signals as occurs under the experimental conditions, which will be described below, represents regenerative phenomena and is almost diagnostic of CICR in β-cells. When a compound elicits periodic amplification of Ca²⁺ signals by itself or enhances periodic amplification of Ca²⁺ signals elicited by the specific experimental protocols, which will be described hereinafter, it is likely that the compound is acting by sensitizing CICR and that the compound may stimulate insulin secretion in a context-dependent manner by targeting CICR.

In order to confirm CICR and to identify which channels are involved in CICR, compounds known in the art to affect IP3R, RyR or the ER Ca²⁺ pump can be used. For example, confirmation that the periodic amplification of Ca²⁺ signals is due to CICR can be obtained by using low concentrations of compounds such as, for example, thapsigargin, caffeine or dantrolene. When periodic amplification of Ca²⁺ signalling is due to CICR, it disappears or is markedly reduced when thapsigargin (500 nM to 1 μM) is applied to the cells through superfusion systems, while the [Ca²⁺]ᵢ is being continuously recorded on-line. When periodic amplification of Ca²⁺ signalling is due to CICR, it disappears or is markedly reduced when ruthenium red (10 μM) or its related compounds (e.g. ruthenium amine binuclear complex, Ru-360) or dantrolene (75 μM) or its congeners (GIF-0185, GIF-0082, azumolene, aminodantrolene) is applied to the cells through superfusion systems, while the [Ca²⁺]ᵢ is being continuously recorded on-line. When periodic amplification of Ca²⁺ signalling is due to CICR, it increases in magnitude and/or in frequency when caffeine (0.5 mM to 2.5 mM) is applied to the cells through superfusion systems, while the [Ca²⁺]ᵢ is being continuously recorded on-line.

Choice of β-cells for Use in the Method

For screening of compounds that stimulate insulin secretion by targeting CICR, it is preferred to use β-cells obtained from mice, rats or human pancreas or insulinoma cell lines.

It is convenient to use β-cells obtained from ob/ob mice since they have large islets and almost 95% of cells in the islets are β-cells. However, β-cells obtained from some colonies of ob/ob mice may lack large number of ryanodine receptors. When cells obtained from other mice, rats or human islets of Langerhans are used, one needs to establish that the cell being examined is likely to be a β-cell. This can be examined by applying tolbutamide (40 μM) or glucose (10 mM) to the cell through perfusion while [Ca²⁺]ᵢ is being recorded simultaneously. If the cell does not respond by elevation of [Ca²⁺]ᵢ when exposed to tolbutamide or glucose, it is unlikely to be a β-cell and must not be used for screening of CICR-activating agents. In general, β-cells are larger than non-β-cells and selection of large cells that respond to glucose (10 mM) or tolbutamide (100 μM) by an elevation of [Ca²⁺]ᵢ makes likely that a β-cell is being examined.

In the present method, it is generally preferred to use β-cells having ryanodine receptors, especially since CICR involving the RyR has been found to be more pronounced. Thus, depending on the specific β-cells to be used, it may be desirable to examine whether said cells have ryanodine receptors. This can be accomplished by testing the effect of caffeine on [Ca²⁺]ᵢ according to the protocols of Islam MS et al in In situ activation of the type 2 ryanodine receptor in pancreatic beta cells requires cAMP-dependent phosphorylation, Proc.Natl.Acad.Sci.U.S.A., 95, 6145-6150 (1998).

Insulinoma cell lines obtained from rat, mouse or hamster can be used for screening CICR-active agents. In this respect the cell lines that are glucose-responsive, i.e. that respond to glucose by elevation of [Ca²⁺]ᵢ, or by stimulation of insulin secretion are more useful. Furthermore the cells should respond to the physiological range of changes in glucose concentration. Typically, a check for insulin secretion response to a change in the glucose concentration from 3 mM to 10 mM is sufficient. Often the cells also respond to a change in the glucose concentration from 3 mM to 7 mM. If insulinoma cells do not respond to glucose, they should not be used for screening for CICR-active agents.

The insulinoma cells can be one of the following: INS-1 cells, various glucose-responsive clones derived from INS-1 cells, betatc3 cells, MIN6 cells and some clones of HIT cells.

A new type of β-cell, called the S5 cell, which is particularly suitable for use in screening CICR-active drugs in β-cells has been developed by the present inventor. The S5 cells can be obtained from INS-1E cells by adapting the latter to culture conditions where fetal bovine serum is reduced to 2.5% and 2-mercaptoethanol is increased to 500 μM. The S5 cells are more differentiated, more slowly growing than ordinary insulinoma cells, and performs more like normal β-cells.

The S5 cells used in the examples below were obtained by adapting the cells to the above culture conditions over 37 passages. These cells were cultured in RPMI 1640, with L-Glutamine (Life Technologies: Catalog. No. 21875-034), supplemented with HEPES (10 mM, Life Technologies. Catalog. No. 15630-049), and sodium pyruvate (1 mM, Life Technologies. Catalog No. 11360-039). 2.5% (v/v) heat-inactivated fetal bovine serum was added to this medium and the medium was aliquoted in 50 ml tubes. To this medium, 500 μM 2-mercaptoethanol (Life Technologies. Catalog. No. 31350-010) and penicillin (50 i.u./ml), streptomycin (50 μg/ml) (Life Technologies. Catalog. No. 15070-063) was added immediately before the medium was used for culture. 2-mercaptoethanol (50 mM, stock) was aliquoted and stored frozen in 100 μl portions and was thawed just before use.

A desired set of β-cells to be used in the inventive method can be obtained by means of any suitable method known in the art. A set can for example conveniently be obtained by culturing a number of β-cells, such as for example the above-mentioned S5 cells.

β-cells can for example be seeded directly on to sterile glass coverslips at a concentration of about 50000 cells per ml in RPMI-1640 medium with hepes, pyruvate, 2mercaptoethanol and FBS. It is preferred not to use poly L-lysine, collagen or extra-cellular matrix for attachment of cells on to the coverslips because such arrangements make the cells abnormally flat and alter their nano-architecture. A drop of medium containing the cells is put on the center of the coverslip and spread gently with a pipette tip. The coverslip, which is placed in a small petridish and left in a humidified CO₂ incubator at 37° C. for 30 min to 2 hrs. When insulinoma cell lines are used 30 min is enough for attachment to the glass coverslips. Primary β-cells from rodent or human islets are allowed to attach for 1-2 hrs. Attention is needed to check that the medium does not get dried up during this incubation. After 30 min to 2 hrs for attachment the petridishes are brought out of the incubators and 2-3 ml warm (37° C.) culture medium is added very gently to the dishes. Attention is needed to see that the coverslips do not float. The cells are suitably cultured for two days before using them for experiments.

Depending on the mode of detection of CICR in the claimed method different Ca²⁺ indicators can be used. The mode of detection is not critical to the invention as long as CICR is not unduly disturbed thereby. A presently preferred mode of detection is by means of fluorescence, in which case a fluorescent Ca²⁺ indicator is used.

It is preferred that the loading of the cells with a given Ca²⁺ indicator is such that just enough fluorescence signal from the β-cells for detection purposes (e.g. 5000 to 10000 cps) can be obtained. Very low level of loading is necessary since high loading interferes with Ca²⁺ homeostasis and alters metabolic state of the β-cells. Accordingly, a highly purified grade of Ca²⁺ indicator is preferably used. As a suitable example, fura-2 AM, FluoroPure grade in special packaging, from Molecular probes (catalog no. F-14185) or Fura-4F AM (molecular probes) can be used, such as in the following example. Similar indicators from other manufacturers may be also suitable.

In order for the cells to be able to effect CICR, the intracellular store of calcium should be filled. This can be accomplished by treating the cells with a high glucose containing solution. In order to keep the cells relatively quiet a concentration of about 3 mM can be used. When higher concentrations are used the cells will become stimulated. However, by adding diazoxide higher glucose concentrations, such as 10-12 mM, can be used while the cells still are kept quiet and the base line [Ca²⁺]ᵢ is kept low.

As a specific example of a suitable loading procedure of a fluorescent Ca²⁺ indicator, the following procedure is provided: In this example the cells are loaded in RPMI-1640 medium containing 0.1% bovine serum albumin. The RPMI medium for loading with fura-2 does not contain 2-mercaptoethanol, Hepes or fetal bovine serum, but preferably contains diazoxide 100 μM to keep the β-cells quiet during incubation in the medium, which contains 11 mM glucose. Pyruvate may be omitted from the loading medium. Fura-2 AM concentration for loading is 0.2-1 μM. The concentration should be kept as low as possible for measuring fluorescence. High concentration of fura-2, e.g. 2-5 μM abolishes CICR since at such concentrations, the intracellular fura-2 concentration is also high and it chelates Ca²⁺ that enters through the voltage-gated Ca²⁺ channels. Fura-4 AM and related fura indicators can be used instead of fura-2Thereafter, the cells on glass cover slips are incubated in fura-2 AM for 35 minutes in the CO₂ incubator at 37° C. and then transferred to a physiological solution containing 11 mM glucose for additional 10 minutes. The composition of this physiological solution (modified KRBH) is: NaCl 140, NaHCO₃ 2, KCl 3.6, NaH₂PO₄ 0.5, MgSO₄ 0.5, HEPES 10, CaCl₂, 1.5, BSA 0.1%. This physiological solution for loading with Ca²⁺ indicators also contains diazoxide 100 μM. The diazoxide should preferably be “Hyperstat” (Schering Plough) or similar preparation that maintains diazoxide in aqueous solution. The stock solutions of diazoxide should be carefully examined for precipitation of diazoxide crystals and when this occurs the solution should be discarded. Diazoxide should preferably not be dissolved in dimethyl sulfoxide (DMSO) for these experiments. Diazoxide may be omitted during loading of Ca²⁺ indicators but in that case the last ten minutes incubation in the physiological solution should contain 3 mM glucose. If the cells are loaded with Ca²⁺ indicators in medium containing 3 mM glucose for 45 minutes, the cells may not show CICR since under such conditions the endoplasmic reticulum Ca²⁺ stores become relatively empty.

Instrument Set Up and Procedures for Experiments for Detecting CICR in β-cells

Suitable instrumentation set up will of course be dependent on the mode of detection of CICR selected. The following is a specific example of set up and procedure for fluorescence detection.

In the following example amplitude of fluorescence signals from single β-cells was measured using a microscope-based fluorescence system (Olympus microscope and a M-39/2000 RatioMaster fluorescence system (PhotoMed). Microscope-based fluorescence systems from other manufacturers can also be used. The cells were excited at wavelengths of 340 nm and 380 nm, and emitted light selected by a 510 nm filter was monitored by a photomultiplier. Excitation wavelengths, 340 nm and 380 nm are generated alternately by a fast monochromator. Fluorescence data are acquired at a rate to yield two ratios per second. Cells on the coverslip are mounted as the bottom of a temperature controlled chamber (RC-2 1BRW chamber and PH2 platform, Warner instruments inc., U.S.A). The temperature of the fluids in the perfusion chamber is maintained at 37° C. by a probe placed in the chamber. The temperature of the fluids in the chamber is monitored continuously. If this temperature is not maintained CICR may not be elicited. The fluid is continuously perfused at a rate of 2 mL per minute using a peristaltic pump and the solution is heated just before it enters into the chamber by an on line solution-heater (Warner instruments, Inc.). The rate of solution exchange should be fast.

The optics of the system should be of high quality to allow optimal transmission of light and detection of fluorescence signals. In the example a 40×oil immersion objective with 1.35 numerical aperture (Uapo/340, Olympus) was used. This objective allows high transmission of 340 and 380 wavelengths. Microscope objectives of similar or better quality can be used.

At the beginning of fluorescence experiments, the cells are inspected in the microscope to choose the “best-looking” cells. Usually, these are relatively large cells with intact margin and the cells are not very flattened. In a case where S5 cells are used, cells that are round and do not have neuron-like processes should be used. These are more differentiated and responds by CICR more often. A small area that includes the cells is chosen for measuring fluorescence from that area.

Fluorescence is preferably recorded from single β-cells. If a large group of cells or an islet is used, CICR is difficult or even impossible to identify.

As will be described below fluorescence imaging systems can be used instead of photomultiplier-based systems, for detecting the periodic amplification of Ca²⁺ signals and thereby CICR, but are presently less preferred. In such a case images are to be acquired at a rate sufficient to yield two ratio points per second. When imaging systems are used, it is not good to use only the brightly fluorescent cells. These cells contain high amount of Ca²⁺ indicators inside the cytoplasm, which buffers incoming Ca²⁺ and makes CICR impossible, and one can not be sure that the changes seen in these images are due to CICR.

### Detection of CICR by Fluorescence Imaging Techniques

Fluorescence imaging systems can be used to detect CICR from β-cells. Ratiometric Ca²⁺ indicators like fura-2 are loaded into β-cells as described before. Images can be taken from a field that contains about 10-30 discrete cells. It is likely that such a field will contain some cells that are capable of responding by CICR. Images should be collected fast so that at least 1-2 ratios per second can be obtained. This is not a limitation since fast speed fluorescence cameras can take pictures at a rate as high as 60 per second. Signals should be stored as raw data and should not be averaged. Data should be collected for about 5 minutes after addition of the test substances. Later on signals from all of the individual cells are analysed by analysing the images. This can be done by appropriate image analysis software. If any of the cells in the field show periodic amplification of Ca²⁺ signals then the test is positive.

The imaging technique can be adapted to high throughput screening systems. In this case the cells are plated in multiwell plates at low dilutions so that discrete single cells are present in the field. Images are first taken before addition of the test substances. After addition of the test substances in the multiwell plates images are collected again for about five minutes. The wells are imaged at a fast rate to obtain at least 1-2 fluorescence ratios per second. This will generate a large amount of data which has to be handled with appropriate storing and computing facilities. Fluorescence signals from at least 5-10 single cells from each wells should be analysed at if any of them shows periodic amplified Ca²⁺ signals the test is regarded as positive. Appropriate softwares can be used for image analysis from the multiwell plates.

### EXAMPLES OF EXPERIMENTAL PROCEDURES AND VARIATIONS

The following are examples of different protocols which can be used for addition of a candidate compound to be tested to the β-cells, and for monitoring of periodic amplified Ca²⁺ release in the cell after addition of the candidate compound.

### Example 1 Control Experiment

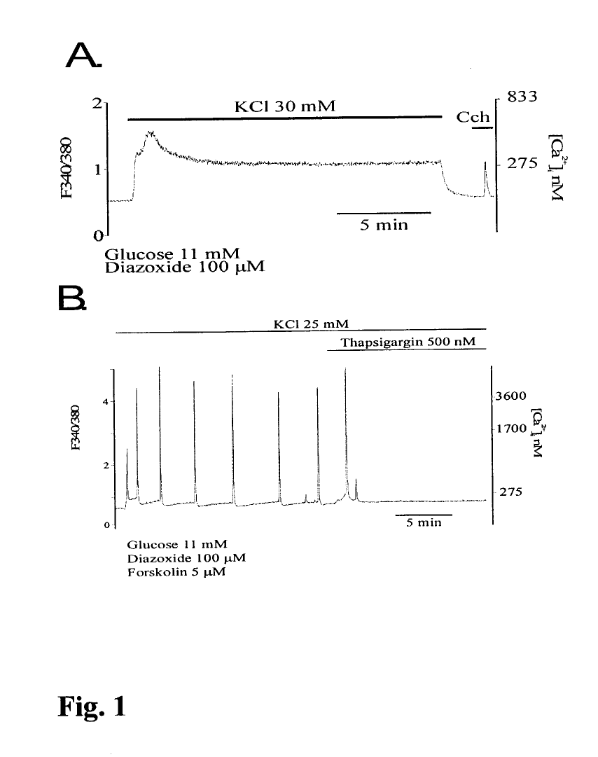

In FIG. 1A, a control experiment according to the following protocol is shown. The cells in the chamber are perifused with the physiological solution containing 11 mM glucose and 100 μM diazoxide (solution A). The [Ca²⁺]ᵢ at the beginning of the experiment is about 35-100 nM. This can be estimated roughly by looking at the 340 and 380 signals. If resting [Ca²⁺]ᵢ appears to be unusually high the cell should be excluded from experiment and a new cell selected instead. The fluorescence signals should be stable. If the fluorescence signals decline rapidly indicating leakage of fura-2, the cell should be excluded from the experiment and a new cell or a new coverslip chosen for experiment. Such leakage of Ca²⁺ indicators or high basal [Ca²⁺]ᵢ are signs of poor health of the cells and such cells are not suitable for use in screening for CICR-active agents. When fluorescence is stable (usually 30 s to a few minutes), the solution is changed to one containing 30 mM KCl in addition to glucose (11 mM) and diazoxide (100 μM) (solution B). Equimolar concentration of NaCl is reduced from this solution. On addition of this solution [Ca²⁺]ᵢ goes up first rapidly and then slowly to a peak. After about 3 min, the KCl-containing solution and [Ca²⁺]ᵢ then returns to normal within a few minutes. The experiment represents a control experiment for a subsequent CICR-screening experiment. At the end of this, the cell is discarded. Prolonged use of one cell in such experiments makes it difficult or impossible to detect CICR.

### Example 2

A new cell from a new coverslip is taken for experiment and is perifused with solution A, which in addition contains a test substance that is likely to trigger CICR. The procedures described for the control experiment are repeated for the test cell, and at an appropriate point of time solution B containing in addition the test substance is added. In FIG. 1B, this is shown for the substance forskolin (5 μM). In a typical positive response [Ca²⁺]ᵢ first goes up to a level and then goes up even further in the form of a large and transient spikes, which then returns to an elevated level of [Ca²⁺]ᵢ. This is a form of amplified Ca²⁺ signal. Following this there is then a series of amplified Ca²⁺ signals appearing at variable intervals. The initial amplified Ca²⁺ signal may be missing in some responses but still the subsequent periodically amplified Ca²⁺ signals are present. The response is characterized by following properties: 1large [Ca²⁺]ᵢ increase; 2transient [Ca²⁺]ᵢ increase (a few seconds in duration); 3regeneratative; and 4periodic, i.e. appearing at intervals of a few seconds to a few minutes. As a minimum, the test substance should give at least one such amplified Ca²⁺ signal. The experiment is continued for about 5 minutes and can often be continued for 30 minutes.

During prolonged experiments, solutions containing pharmacological tools can be added to determine whether the CICR is mediated by inositol-1,4,5-trisphosphate receptor or RyR, and to confirm whether it is CICR or not. Such tools include, caffeine, isocaffeine, ryanodine, dantrolene, xestospongin C, 2-aminoethoxydiphenyl borate (also called diphenylboric acid 2-aminoethyl ester), thapsigargin, cyclopiazonic acid, 2,5-di-(tert-butyl)-1,4-benzohydroquinone, ruthenium red of thapsigargin, eudistomin D, bastadins, U73122 (1-[6-([(17b)-3-methoxyestra-1,3,5(10)-trien-17-yl]amino)hexyl ]-1H-pyrrole-2,5-dione), ET-18-OCH3 (1-octadecyl-2-methyl-rac-glycero-3-phosphocholine) or related compounds.

### Example 3

Example 2 was repeated, except that the cells were perifused first with solution A and then with solution B. After [Ca²⁺]ᵢ had increased to a plateau level by solution B, a new solution B containing in addition the test substances was added. The new solution B containing the test substance may then give rise to the periodic amplification of Ca²⁺ signals, if the substance is a CICR active agent.

### Example 4

In this example a substance known to elicit CICR reproducibly first elicits the periodic amplification of Ca²⁺ signals. Such a substance can for example be forskolin (2.5-5 μM). The cell is first perifused with solution A, then with solution B containing forskolin. The periodic amplification of Ca²⁺ signals and their frequency are first noted. A third solution containing solution B and a test substance (in addition to forskolin) is then added. If this increases the frequency or amplitude of the periodic amplification of Ca²⁺ then the substance is a sensitizer or activator of CICR. If the test substance decreases the frequency or amplitude of the periodic amplification of Ca²⁺ then the substance is an inhibitor of CICR.

### Example 5

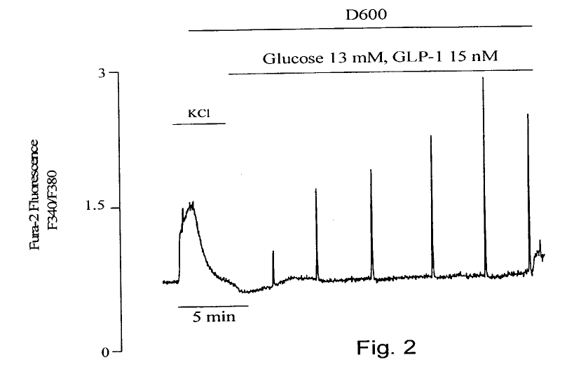

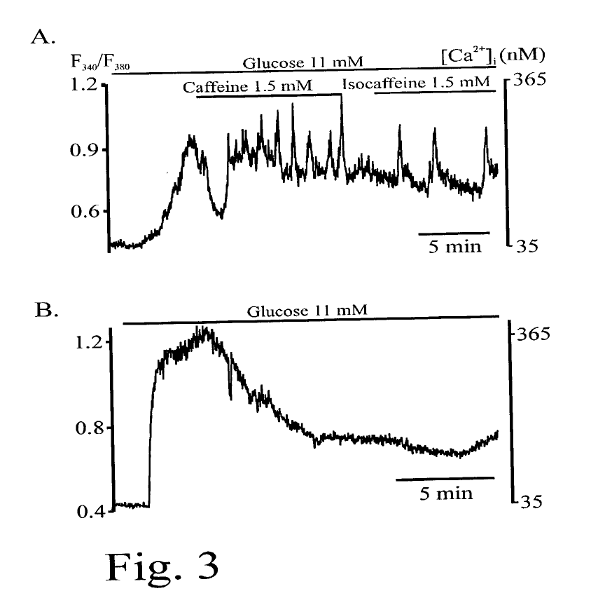

In this example, yet another variation is described. With reference to FIG. 2, the test substance, glucagons-like peptide 1, gave rise to periodic amplification of Ca²⁺ signals.

The cell is perifused with the physiological solution containing 3 mM glucose. The cell is then depolarized by 30 mM KCl in the same physiological solution. After [Ca²⁺]ᵢ goes up (due to Ca²⁺ entry through L-voltage-gated Ca²⁺ channel), 10 μM D600 or verapamil is added to the solution. [Ca²⁺]ᵢ then returns to near base line. At such concentrations, D600 or verapamil does not completely block the L-type Ca²⁺ channels but reduces frequency of opening of the L-type Ca²⁺ channels. This reduces the component of [Ca²⁺]ᵢ that is due to Ca²⁺ entry through L-type Ca²⁺ channels and thus allows the CICR component to be visualized better. After D600, when [Ca²⁺]ᵢ returns to the base line, a new solution containing 13 mM glucose and the test substance (in addition to KCl and D600) is added. If the test substance sensitizes or activates CICR, there appear periodically amplified Ca²⁺ signals. Alternatively, a known CICR-sensitizing agent under these experimental conditions first elicits CICR, and thereafter a solution containing unknown test substance is added to see if it increases or decreases frequency and/or amplitude of the periodic amplification of Ca²⁺ signals.

### Example 6

Another variation that works with some insulin-secreting cells and some tests is as follows. The cell (β-cells, which are preferably S5 cells) is first perifused with physiological solution containing 3 mM glucose and then with the same solution containing 10 mM glucose. A solution containing 10 mM glucose and the test substance is then added (see FIG. 3). This will induce periodic amplification of Ca²⁺ signals indicating that the substance is a sensitizer or activator of CICR. As can be seen in FIG. 3, the test substances caffeine and isocaffeine gave rise to periodic amplifications of Ca²⁺ signals. Caffeine was more potent than isocaffeine and accordingly caffeine gave more frequent CICR. A is the test and B is the control experiment.

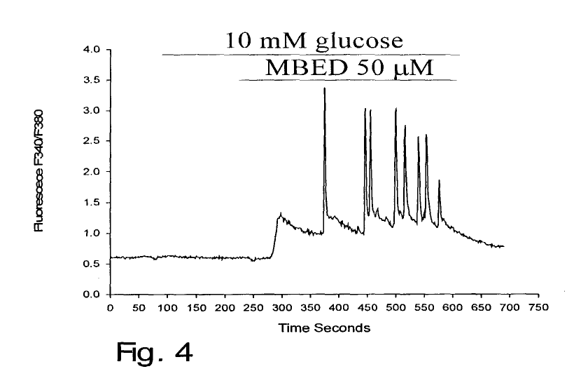

In FIG. 4, same experiment is shown, except for that MBED and S5 cells were used.

### Example 7

Other variations of the method for screening CICR-active agents in β-cells are possible wherein the filling state of the ER, the phosphorylation status of the cell, and/or sensitivity of the intracellular Ca²⁺ channels is increased by known pharma-cological probes, such as, e.g. caffeine, cAMP, nitric oxide, cyclic ADP ribose or fructose 2,6-biphosphate.

In all the above protocols the relative potency of an CICR-active agent can be estimated semi-quantitatively from the frequency and amplitude of the amplified Ca²⁺ signals.

### Compounds identified by means of the above screening method-Insulin secretion, Mechanisms and Receptors Involved

By means of the screening method the compounds MBED, caffeine (to a lesser extent iso-caffeine) and forskolin have been found to elicit periodic amplified Ca²⁺ release in β-cells. However, as opposed to the other compounds, forskolin does not bind directly to the RyR, but act through phosphorylation. Subsequent testing of the compounds has also confirmed context dependent stimulation of insulin secretion in β-cells by said compounds.

Compounds falling within the general definition given by the following general formula are also expected to exhibit similar activity due to structural and chemical similarity:

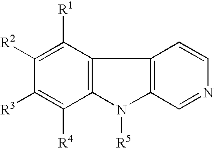

wherein,

R¹ is a halogen atom;

R² is a hydroxyl, methoxy or acetoxy group;

R³ is hydrogen or a halogen atom

R⁴ is hydrogen or an acetoxy group; and

R⁵ is hydrogen or a methyl group,

with the proviso that at least one of R⁴ and R⁵ is hydrogen.

Suitable compounds according to the present invention are for example those described in JP-2579789, and by Asami Seino-Umeda et al. in J. Pharn. Pharmcol. (2000), 52: 517-521, and by Kobayashi, J., et al. in J. Pharn. Pharmcol. (1988), 40: 62-63.

A generally preferred group of compounds according to the present invention are those wherein R² is a hydroxyl group, and more preferably the derivatives of eudistomin D, i.e. wherein R² is a hydroxyl group and R¹ is bromine.

In compounds wherein R¹ and R³ both are halogen atoms, and especially in such compounds wherein R² is a hydroxyl group, it is generally preferred that R¹ and R³ are the same, suitably iodine, chlorine or bromine, more preferably chlorine or bromine, and most preferably bromine.

In compounds wherein R² is methoxy, R⁴ is preferably acetoxy.

In compounds wherein R² is acetoxy, R⁴ is preferably hydrogen.

Specific examples of compounds of the present invention are 8-acetoxy-5-iodo-6-methoxypyrido[3,4-b]indole, 5,7-dibromo-6-hydroxypyrido[3,4-b]indole (also referred to as 7-bromoeudistomin D), 5,7-dibromo-6-acetoxypyrido[3,4-b]indole, 5,7-dibromo-6-acetoxy-9-methylpyrido[3,4-b]indole, 5,7-dibromo-6-hydroxy-9-methylpyrido[3,4-b]indole (also referred to as 9-methyl-7-bromoeudistomin D or MBED), 5,7-dichloro-6-hydroxypyrido[3,4-b]indole, 5,7-dichloro-6-hydroxy-9-methylpyrido[3,4-b]indole, 5,7-diiodo-6-hydroxypyrido[3,4-b]indole, and 5,7-diiodo-6-hydroxy-9-methylpyrido[3,4-b]indole.

Examples of preferred compounds are 8-acetoxy-5-iodo-6-methoxypyrido[3,4-b]indole, 9-methy-7-bromoeudistomin D, 5,7-dibromo-6-acetoxypyrido[3,4-b]indole, 5,7-dibromo-6-acetoxy-9-methylpyrido[3,4-b]indole, 5,7-dibromo-6-hydroxy-9-methylpyrido[3,4-b]indole, 5,7-dichloro-6-hydroxypyrido[3,4-b]indole, and 5,7-diiodo-6-hydroxypyrido[3,4-b]indole.

Examples of more preferred compounds are 8-acetoxy-5-iodo-6-methoxypyrido[3,4-b]indole, 9-methyl-7-bromoeudistomin D and 7-bromoeudistomin D, of which compounds 9-methyl-7-bromoeudistomin D is the most preferred.

MBED is highly lipophilic potent and effective in stimulating insulin secretion from beta-cells in a context dependent manner. The mechanism underlying this distinct effect of MBED involves the ryanodine receptor and CICR, as evidenced from the fact that known activators of ryanodine receptors, like caffeine, also stimulated context dependent insulin secretion. Such context dependent insulin secretion was not due to inhibition of beta-cell cAMP-phosphodiesterases or inhibition of adenosine receptors. Accordingly, while not wishing to be bound to any theory, the present inventor concludes that the β-cell ryanodine receptor and CICR are distinct targets for developing drugs that would stimulate insulin secretion in a context dependent manner and that MBED represents a prototypic compound for developing such therapeutic agents.

In the following, testing for stimulation of insulin secretion by molecules found to elicit periodic amplified in the screening method will be described, , and more particularly with reference to MBED. The underlying mechanism of action and receptors involved will also be described in the following experiments.

### Materials used

INS-1E rat insulinoma cells were from C. B. Wollheim and P. Maechler, Geneva. Caffeine, isocaffeine, glucose (Sigma, G-5146) and dantrolene were from Sigma. 3,9-dimethylxanthine was from Fluka. Ryanodine and thapsigargin were from Calbiochem. 9-methyl-7-bromoeudistomin D hydrochloride (MBED) was from Dr. Guy Nadler, SmithKline Beecham, France. ³H-cyclic AMP was from Amersham. Rat insulin ELISA kit was from Mercodia AB, Sweden. ¹²⁵I-insulin was from Euro-Diagnostica AB, Sweden.

### Experimental Methods Used

Cell Culture:

Glucose-responsive clonal insulinoma cells (INS-1E) were cultured in RPMI-1640 medium supplemented with FBS (5%, v/v), penicillin (50 i.u. /ml), streptomycin (50 μg/ml), 2-mercaptoethanol (50 μM), HEPES (10 mM) and sodium pyruvate (1 mM) (Maechler, P. et al., IUBMB.Life 50, 27-31(2000). Medium was changed every other day.

Insulin Release From Cells:

INS-1E cells (200,000/ well) were seeded in 24-well plates and cultured for 6-7 days before using them for insulin release assay. On the day of experiment, cells were incubated in RPMI without glucose for 2 hours. Cells attached to the wells were then washed three times with warm (37° C.) medium (KRBH) containing (in mM): NaCl 140, NaHCO₃ 2, KCl 3.6, NaH₂PO₄ 0.5, MgSO₄ 0.5, HEPES 10, CaCl₂, 1.5, BSA 0.1% and incubated for 30 minutes at 37°. Cells were then incubated with 500 μl of the test solutions, by adding solution to one well at a time, every 20 sec. After one hour of incubation, 200 μl of supernatant was transferred to Eppendorf tubes, again one well at a time, every 20 sec. The collected materials were then centrifuged and supernatants were used for insulin ELISA.

Islet Preparation

Normal lean mice (BALB/c, Bomholtgard, Ry, Denmark) weighing 20-25 g were starved overnight and islets isolated by collagenase digestion and dextran purification method (Shi, C. L., Cell Transplant. 6, 33-37 (1997)). Islets were then cultured overnight in RPMI 1640 medium containing 10% FBS, 11.2 mM glucose, 60 i.u./ml penicillin and 60 μg/ml gentamicin, in a water-saturated atmosphere of air and 5% CO₂.

Insulin Release From Islets

Groups of 50 islets were transferred to a perifusion chamber housed in an infant incubator at 37° C. They were perifused at a rate of 1 ml/ min with Krebs-Ringer bicarbonate buffer supplemented with 20 mM HEPES, 0.1% BSA, glucose and drugs as required. The buffer was continuously gassed with 95% 02 and 5% CO₂. The islets were perifused for 30 min with this medium containing 2 mM glucose, followed by a 40-min stimulation with 11.2 mM glucose alone (control), or 11.2 mM glucose plus drugs. During the last five minutes of perifusion with 2 mM glucose, three samples of effluent were collected to measure basal rate of insulin secretion. After switching to the 11.2 mM glucose the effluent was sampled every minute for the first five minutes and then at the intervals of 5 minutes. The average rate of insulin secretion (ng/min/50 islets) was calculated by integrating individual perifusion profile. Insulin was measured by radio immunoassay using crystalline mouse insulin as standard (Shi, C. L. cited above).

Phosphodiesterase Assay:

Rat insulinoma cells were detached from the flasks by trypsin-EDTA. The cells were homogenized in Tris buffer containing (in mM): Tris 10, sucrose 250, EDTA 1.0, phenylmethylsulphonyl fluoride 0.01 and benzamidine 1.0The homogenates were centrifuged (48,000 g, 20 min, 4° C.). Both soluble extracts and the pellet were assayed for cAMP-PDE activity with 0.5 μM 2,8-[³H]-cyclic AMP as substrate (see Ahmad, M. et al., Br.J.Pharmacol., 129, 1228-1234 (2000) and Thompson, W. J. and Appleman, M. M., Biochemistry, 10, 311-316 (1971)). Assays were performed by the two-step radiometric assay under linear rate formation of product and where less than 10% of substrate is utilized. Activity was expressed as pmol/min/ml and percentage inhibition of the control value.

Measurement of cAMP Content

INS-1E cells (20000 per well) were cultured for three days in 24 well plates. On the day of the experiments cells were washed and incubated for 30 min at 37° C. in the KRBH buffer containing 3 mM glucose. Cells were then washed again and solutions containing 11.2 mM glucose and test substances were added. After 10 min, incubations were terminated by removing the medium and precipitating cell protein in 80% ice-cold ethanol. Cells were then scraped and all contents of the wells were transferred to tubes. After centrifugation at 14,000 rpm for five min, the supernatants were collected and freeze-dried over-night. The cAMP content in each sample was determined with a radio ligand-binding assay using a bovine heart cAMP-binding protein as described by Pyne, N. J. and Tolan, D., Pyne, S. in Biochem.J 328 (Pt 2), 689-694 (1997).

Confocal Microscopy Imaging:

INS-1E cells were incubated in medium containing 5 mM glucose and 5 μM fluo-3 AM for 30 minutes followed by 10 min in medium without fluo-3Cover slips were mounted in a superfusion chamber, which was superfused with physiological solution at 3 ml/min. A three-way solenoid valve system allowed for rapid exchange of solutions. The dead space of the system (from valve inlet to chamber) was -0.5 ml. The flow was increased to 6 ml/min 15 s prior to switching from control solution to experimental solutions and reduced back to 3 ml/min after return to normal solution. [Ca²⁺]₁ were recorded with a BioRad laser scanning confocal system (BioRad MRC 1024) attached to a Nikon Diaphot 200 inverted microscope equipped with Nikon Plan Apo 60×1.4 NA oil immersion objective. Fluo-3 was excited at 488 nm (15 mW krypton-argon mixed gas laser, intensity attenuated to 3%) and the emitted light was collected through a 522 nm narrow band filter. Images were obtained with the iris closed to the minimum size that was compatible with good image quality. For each image, 3× Kalman averaging was used. Images were stored and converted to pseudo-color images using Scion Image software (Scion Corporation, Maryland).

Ratios were calculated using the initial fluorescence F₀, and the observed fluorescence F using the equation R=F/F₀, and these ratios were subsequently converted into [Ca²⁺]ᵢ using the equation: [Ca²⁺]ᵢ=RxK_{D}/((K_{D}/resting [Ca²⁺]ᵢ)−R). K_{D}, the apparent dissociation constant of fluo-3 at 25 ° C., was taken to be 480 nM. Resting [Ca²⁺]ᵢ in these cells under the conditions of experiment was 35 nM. All experiments were carried out at room temperature (25° C.).

### Measurement of [Ca²⁺]ᵢ by Microfluorometry

Mouse β-cells plated on glass cover slips were incubated in RPMI 1640 medium containing 0.1% bovine serum albumin and 0.6 μM fura-2AM for 30 min. Cells were then incubated for an additional 10 min in the basal medium containing 3 mM glucose. [Ca²⁺]ᵢ was measured as described previously with the modifications that an Olympus microscope and a M-39/2000 RatioMaster fluorescence system (PhotoMed) were used (Islam, M. S., et al. in Proc.Natl.Acad.Sci.U.S.A 95 (11):6145-6150 (1998) cited above).

The following experiments were carried out using the above described methods and materials.

### EXPERIMENTS

### 1. Effect of MBED on Insulin Releasefrom INS-1E Cells

A. Stimulation

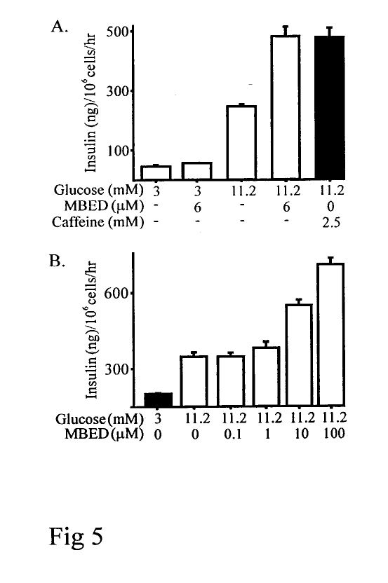

In this experiment, the effect of MBED on insulin secretion from a highly differentiated clonal insulin secreting cell line INS-1E was studied. MBED (6 μM) was applied in the presence of 3 mM or 11.2 mM glucose. For comparison, the effect of caffeine (2.5 mM) on insulin secretion from the same cells was also studied. The results are presented in FIG. 5A.

From FIG. 5A, it can be seen that MBED released insulin in a context-dependent manner. There was no stimulation of insulin secretion in the presence of 3 mM glucose, but marked stimulation occurred in the presence of 11.2 mM glucose. The effect of MBED on stimulation of insulin secretion in the presence of 11.2 mM glucose was conspicuous: 6 μM MBED almost doubled the insulin secretion (FIG. 5A) and the response was similar to that obtained with caffeine (2,5 mM), a well-known stimulator of the ryanodine receptor. Accordingly, it can be seen that MBED is potent and effective in stimulating insulin release from INS-1E cells in a glucose-dependent manner.

B. Dose-response of MBED

In this experiment the dose response of MBED induced insulin secretion was investigated. INS-1E cells were cultured in 24 well plates. Insulin release and insulin assay was performed as described above. MBED was tested at a concentration of 0.1 to 100 μM in the presence of 11.2 mM glucose. The most commonly used activator of ryanodine receptor in β-cells is caffeine. Caffeine is however only effective when used at high concentrations. From FIG. 5B it can be seen that MBED stimulated insulin secretion in a does-dependent manner and was in this respect about 400 times more potent than caffeine (FIGS. 5A and 5B).

### 2Stimulation of Insulin Secretion From Islets by Caffeine and MBED

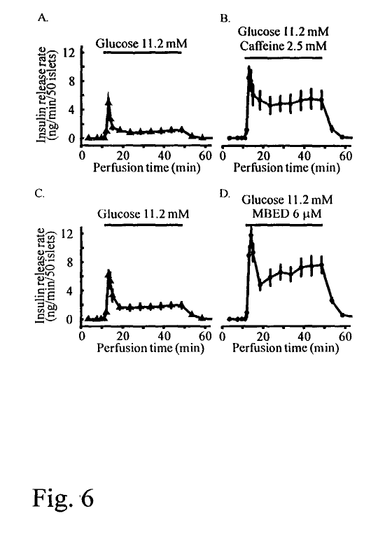

Glucose-responsive insulin secreting cell lines have been widely used to study stimulus-secretion coupling in beta cells. However, they may differ from native beta cells in a number of aspects. We therefore tested the effects of MBED on insulin secretion from primary beta cells of mouse islets. The results are shown in FIG. 6. Accordingly, 50 islets from normal lean mice were perfused with physiological solutions containing 2 mM glucose. At times indicated by horizontal bars, the islets were perifused with 11.2 mM glucose with or without sensitizers of RY receptors i.e. 2.5 mM caffeine (B) or 6 μM MBED (D). Caffeine and MBED persistently stimulated both the first- and second-phases of insulin secretion. Each curve represents mean ±SEM of four separate experiments.

As shown in FIGS. 6B and 6D, in the presence of 11.2 mM glucose MBED and caffeine stimulated insulin secretion. The effects of caffeine and MBED on insulin secretion were persistent and the agents increased both the first- and the second-phases of secretion. MBED was clearly more potent than caffeine, the effect of 6 μM MBED being roughly comparable to that of 2.5 mM caffeine. Stimulation of secretion by MBED and caffeine reversed completely on wash out indicating a lack of any major toxic effect. In contrast to their effects on insulinoma cells, isocaffeine and 3,9-dimethylxanthine had no significant stimulatory effect on insulin secretion from mouse islets. The insulin secretion rate (ng/min/50 islets) from control, caffeine-,isocaffeine-, and 3,9-dimethylxanthine-treated islets were 1. 12±0.21, 5.06±1.17 (p<0.032), 1.36±0.35 (p<0.2), and 2.76±0.81 (p<0.1) respectively.

### 3Mechanism of [Ca²⁺]ᵢ Increase in Beta Cells

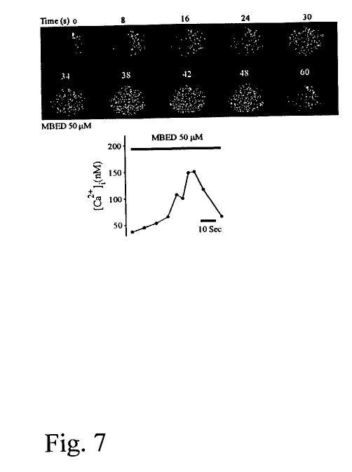

MBED is known to activate ryanodine receptors in different cells (Seino-Umeda, A., Fang, Y. I., Ishibashi, M., Kobayashi, J. & ohizumi, Y. (1998) Eur. JPharmacol. 357, 261-265, and Fang, Y. I., Adachi, M., Kobayashi, J. & ohizumi, Y. (1993) J Biol. Chem. 268, 18622-18625). To test whether the effect of MBED on insulin secretion could be mediated by ryanodine receptors, the effect of MBED on [Ca²⁺]ᵢ in INS-1E cells was tested by confocal imaging of fluo-3 loaded INS-1E cells. 50 μM of MBED was applied to the cells. The resulting images are shown in FIG. 7. The changes in colour represent different degrees of [Ca²⁺]ᵢ increase. From FIG. 7 it can be seen that MBED increased [Ca²⁺]ᵢ in these cells, indicating that the target for MBED-mediated insulin secretion was the ryanodine receptor. After application of MBED (50 μM), the [Ca²⁺]ᵢ increased in the cells first locally, and thereafter increasingly more generally. As can be seen from FIG. 7, and as previously mentioned, imaging is not very good for detecting CICR. At the imaging frequency used in this experiment, one can not be sure that the changes seen in these images are due to CICR. (From FIG. 4, on the other hand, CICR can clearly be identified)

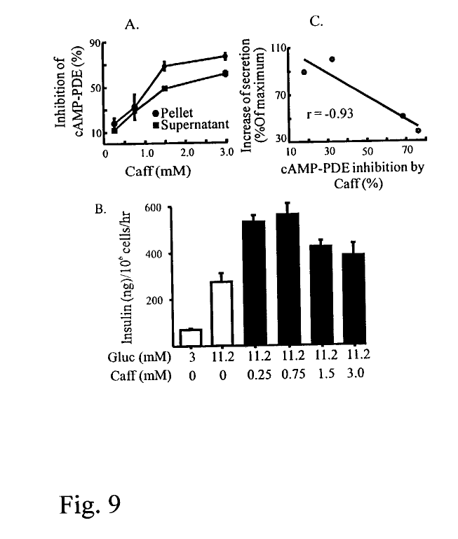

As will be seen below with reference to FIG. 9, MBED was more potent than caffeine, in increasing [Ca²⁺]ᵢ. The MBED-induced increase in [Ca²⁺]ᵢ was slower compared to that observed with caffeine (cf. FIG. 9, upper panel). MBED released Ca²⁺ at localised sites eventually leading to a global increase in [Ca²⁺]ᵢ which returned to baseline in spite of continued presence of the compound.

### 4The Ryanodine Receptor—a Distinct Targetfor Stimulating Insulin Secretion

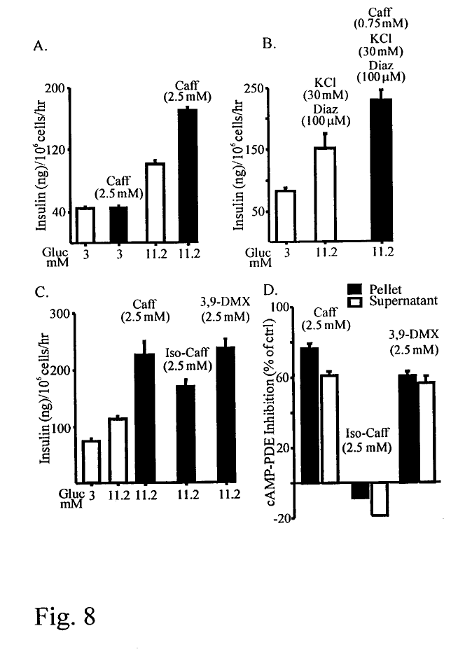

To test whether RY receptors in β-cells are involved in insulin secretion, we tested the effect of caffeine on the INS-1E cells. INS-1E cells were incubated for 1 hr in the presence of low (3 mM) or high (11.2 mM) glucose with or without caffeine. Caffeine (2.5 mM) stimulated insulin secretion in a context-dependent manner (FIG. 8A). It did not alter insulin secretion in the presence of 3 mM glucose but stimulated secretion in the presence of 11.2 mM glucose. Stimulation of insulin secretion by caffeine (0.75 mM) was observed even when the effect of glucose on K_{ATP} channel was bypassed by diazoxide and KCl (FIG. 8B). It may be noted that caffeine induces CICR and increases [Ca²⁺]ᵢ under such conditions. To determine whether the insulin secretion evoked by caffeine was due to sensitization of the RY receptors or could be accounted for by inhibition of cAMP-PDEs alone, we used two caffeine-analogs that have been reported not to inhibit cAMP-PDEs but sensitize the RY receptors. Isocaffeine, a 9-substituted isomer of caffeine (2.5 mM) stimulated insulin secretion significantly (FIG. 8C). Another 9-substituted methylxanthine 3,9-dimethylxanthine (2.5 mM), which sensitizes the RY receptors of β-cells was equally effective as caffeine in stimulating insulin secretion (FIG. 8C). In these experiments, we used methylxanthines at a concentration of 2.5 mM. The effects of caffeine and its two analogs on cAMP-PDE activity in rat insulinoma cells are shown in FIG. 8D. Isocaffeine (2.5 mM) did not inhibit cAMP-PDEs but still stimulated insulin secretion (cf FIG. 8D and C). 3,9-dimethylxanthine (2.5 mM) was slightly less potent than caffeine in inhibiting cAMP-PDEs but was just as effective as caffeine in stimulating insulin secretion (cf FIGS. 8D and C). These results suggest that the effect of caffeine on insulin secretion cannot be fully attributed to inhibition of cAMP-PDEs.

In FIG. 9A, dose response of caffeine on inhibition of cAMP-PDEs in beta-cells is shown. Membrane fractions and supernatants from insulin-secreting cells were tested according to methods described above. Circle represents results obtained from pellet and squares represent those from supernatants. In FIG. 9B, dose response of caffeine on context-dependent insulin secretion is shown. Conditions for experiments were as in FIG. 6C. Caffeine was used at a concentration of 0.25 to 3 mM. At a concentration of as low as 0.25 mM, caffeine caused near-maximal stimulation of insulin secretion (FIG. 9B), while it inhibited cAMP-PDEs by only ˜18%. Maximal inhibition of cAMP-PDEs was achieved with ˜-3 mM caffeine, whereas maximal stimulation of secretion was achieved with only 0.25-0.75 mM caffeine. There was a negative correlation between cAMP-PDE-inhibition by caffeine and stimulation of secretion by the xanthine drug (FIG. 9C) suggesting that a sensitization of the RY receptors might underlie the caffeine-stimulated secretion. Caffeine (0.25-3 mM) did not increase cAMP content in these cells. In the cells that were treated with 0.75 mM caffeine (a concentration that stimulated insulin secretion maximally), cAMP content was 2.29±1. 10 pMol per 20,000 cells, whereas that in the untreated cells was 3.14±1.11 pmol per 20,000 cells (p=0.6, n=4).

### 5. Presence of Ryanodine Receptors in INS-1E Cells

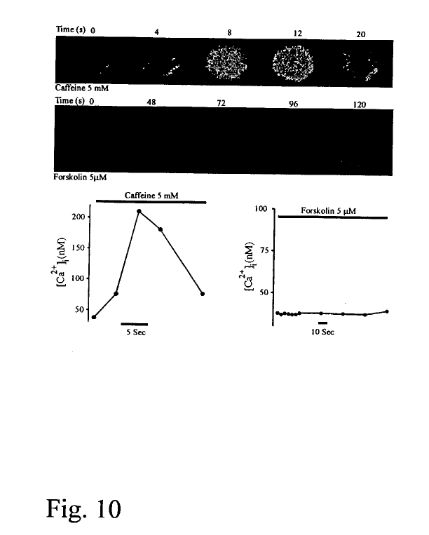

The following experiment was carried out in order to confirm the presence of ryanodine receptors in INS-1E cells. For this purpose, the effect of caffeine on [Ca²⁺]ᵢ was tested. Fluo-3 loaded single cells were imaged by confocal laser scanning microscopy. The results are shown in FIG. 10. Increase in [Ca²⁺ ]i is represented by pseudocolours, where blue indicates minimal and red indicates maximal [Ca²⁺]ᵢ. Upper panel of FIG. 7 shows confocal images of cells stimulated with 5 mM of caffeine. As can be seen, caffeine caused a transient increase on [Ca²⁺]ᵢ, indicating the presence of ryanodine receptors in these cells. The rapid increase of [Ca²⁺]ᵢ by caffeine was global and no clear initiation sites for [Ca²⁺]ᵢ increase by caffeine could be detected. It was concluded that the observed increase in [Ca²⁺]ᵢ by caffeine could not be attributed to inhibition of phosphodiesterases (PDEs) and consequent increase of cAMP, since increase of cAMP by forskolin did not increase [Ca²⁺]ᵢ, as shown in the lower panel.

### 6MBED Does Not Inhibit PDEs

The effects of 9-methyl-7-bromoeudistomin D, (MBED) which sensitises CICR by acting on the caffeine-binding site of RY receptors was tested in this experiment. MBED is not a methylxanthine derivative, and was thus less likely to inhibit PDEs. Membrane or supernatant fractions obtained from insulin-secreting cells were assayed for cAMP-PDE activity in the presence of different concentrations of MBED

TABLE 1
Effect of MBED on cAMP-PDEs in insulin secreting cells.

| | Control | MBED 5 μM | MBED 50 μM |
|---|---|---|---|
| | (pmol/min/ml) | (pmol/min/ml) | (pmol/min/ml) |
| Membrane | 27 ± 3 | 26.5 | 23.2 |
| cAMP-PDE | | | |
| Cytosolic | 33.1 ± 3.6 | 27.7 ± 2.5 | 31.6 ± 0.7 |
| cAMP-PDE | | | |

As shown in table 1, MBED did not inhibit PDEs in INS 1-E cells.

### 9. Ca2+ Release From the Endoplasmic Reticulum is Involved in Dontext-dependent Stimulation of Insulin Secretion

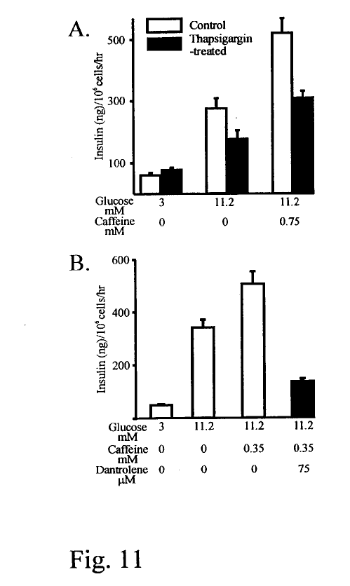

To examine whether release of Ca²⁺ from the ER was involved in stimulation of secretion by caffeine, we tested the effect of caffeine on cells whose ER Ca²⁺ stores were first depleted by prolonged inhibition of SERCA by thapsigargin (FIG. 11A). Basal insulin secretion in Ca²⁺-depleted cells was not different compared to that in controls cells. The glucose-dependent caffeine-stimulated secretion was significantly reduced but not abolished in thapsigargin-treated cells. This suggests that release of Ca²⁺ from the ER by caffeine is one of the mechanisms by which caffeine stimulated insulin secretion in a glucose-dependent manner.

A high concentration of ryanodine is expected to inhibit the RY receptors. However, a 1 hr exposure of the cells to 100 μM ryanodine did not alter the glucose-induced insulin secretion. The insulin secretion rates (ng/10⁶ cell/hr) in control and ryanodine-treated cells were 226±32 and 193±11 (n=4) respectively. The stimulation of secretion by caffeine (0.75 mM) was also not reduced by ryanodine and the insulin secretion rates in control and ryanodine-treated cells were 328±22 and 354±34 respectively. Glucose-dependent stimulation of insulin secretion by caffeine was abolished in the presence of dantrolene (75 μM), an inhibitor of RY receptors (FIG. 11B).

### Discussion

For the first time it is demonstrated that Calcium Induced Calcium Release (CICR) in β-cells is a target for stimulating insulin secretion in a glucose-dependent manner, especially CICR involving the ryanodine receptor. A method of screening for compounds stimulating insulin secretion in a glucose-dependent manner is also described for the first time, which method is based on the finding that such compounds have been found to elicit periodic amplified Ca²⁺ release in β-cells. By means of the method MBED has been found to be a potent insulin secretagogue and its action is special in that it stimulates insulin secretion only when glucose concentration is high. The substance increased insulin secretion from both insulin secreting cell-lines and native islet cells. MBED affected both first and second phase of insulin secretion and was more potent than.caffeine, which is commonly used for studying ryanodine receptors in vitro. The action of MBED involves activation of the ryanodine receptor of beta cells and thereby increase of [Ca²⁺]ᵢ. The action of MBED is not dependent on inhibition of cAMP-PDEs of beta cells as is the case with many methylxanthines. MBED thus represents a novel prototypic drug that uses ryanodine receptor and CICR to stimulate insulin-secretion in a context dependent manner.

In this disclosure the inventor has critically examined the role of ryanodine receptors and CICR process in insulin secretion. For this purpose, caffeine, the classical activator of ryanodine receptors, was initially used. However, an important experimental obstacle for unravelling the mechanisms by which caffeine induces context-dependent insulin secretion has been the inability to sensitise the ryanodine receptor without inhibiting PDEs. These obstacles were circumvented by using an analogue of caffeine that does not inhibit PDEs but activate ryanodine receptors. Furthermore, it has been found that MBED does not inhibit cAMP-PDEs but still stimulates insulin secretion in a context dependent manner pointing to importance of ryanodine receptor and CICR in this process.

Antagonism of adenosine Al receptor can also not explain context dependent increase in insulin secretion since from previous studies it is known that adenosine receptor antagonists do not alter glucose-induced insulin secretion (D. Hillaire-Buys, G. Bertrand, R. Gross, and M. M. Loubatieres-Mariani. Evidence for an inhibitory Al subtype adenosine receptor on pancreatic insulin-secreting cells. Eur.J.Pharmacol. 136 (1):109-112, 1987

Furthermore, unlike methylxanthines, MBED is not an inhibitor of adenosine receptors.

The functional properties of caffeine-and MBED-sensitive Ca²⁺ stores provide clues about their location in the cell. The results suggest that high amplitude Ca²⁺ micro-domains around ryanodine receptors are closely juxtaposed with the insulin secretory granules. In fact these receptors may be situated on insulin secretory granules. The fact that caffeine and MBED potentiate insulin secretion suggests that CICR may occur preferentially at the secretory sites.

In summary, novel potent insulin secretagogues that stimulates insulin secretion in a context-dependent manner have been identified. In particular the very potent lipophilic insulin secretagogue MBED. The molecular mechanisms involved in this process, which involves ryanodine receptor and CICR, has been characterised. The present inventor believes that ryanodine receptor and CICR represent distinct targets for development of new antidiabetic drugs and that MBED represents a proto-typic molecule for further development of therapeutic agents.

As previously mentioned, while in most cases ryanodine receptors mediate CICR, β-cells may however also have other channels that can mediate CICR. Drugs eliciting CICR through such other channels can also be identified by means of the present screening method, which drugs are likely to have the same effect on insulin secretion in β-cells as MBED.

What is claimed is:

1. A method of identifying compounds that stimulate insulin secretion in a context-dependent manner, comprising the steps of:
A. providing a set of β-cells capable of CICR;
B. adding a candidate compound to be tested to the cells; and
C. monitoring the cells for periodic amplified Ca²⁺ release in said cells after addition of the candidate compound of step B.

2. A method of identifying compounds that stimulate insulin secretion in a context-dependent manner, comprising the steps of:
A. providing a set of β-cells capable of CICR;
B. selecting at least one viable/healthy β-cell of said set;
C. adding a candidate compound to be tested to the cell(s) selected in step B; and
D. monitoring said at least one cell selected in step B for periodic amplified Ca²⁺ release in said cell after addition of the candidate compound of step C.

3. The method of claim 1 wherein β-cells having ryanodine receptors are used.

4. The method of claim 1 wherein the β-cells are obtained from ob/ob-mice

5. The method of claim 4 wherein the β-cells are obtained from S5 cells, derived from INS-1E cells.

6. The method of claim 1 wherein the periodically amplified Ca²⁺ release is initiated within 5 minutes from addition of the compound to be tested.

7. The method of claim 1 wherein the monitoring in step C is performed using a fluorescent Ca²⁺ indicator molecule.

8. The method of claim 2 wherein β-cells having ryanodine receptors are used.

9. The method of claim 2 wherein the β-cells are obtained from ob/ob-mice

10. The method of claim 9 wherein the β-cells are obtained from S5 cells, derived from INS-1E cells.

11. The method of claim 2 wherein the periodically amplified Ca²⁺ release is initiated within 5 minutes from addition of the compound to be tested.

12. The method of claim 2 wherein the monitoring in step D is performed using a fluorescent Ca²⁺ indicator molecule.

13. A method of using a compound that elicits periodic amplified Ca²⁺ release in β-cells comprising obtaining a compound identified by the method of claim 1, preparing said compound as a pharmaceutical, and using said compound to treat defective insulin secretion related disorders.

14. The method of claim 13 wherein the defective insulin secretion related disorder is type 2 diabetes.

15. The method of claim 13, wherein the compound is defmed by the following formula 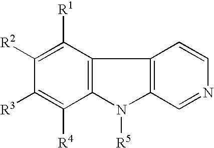
wherein,
R¹ is a halogen atom;
R² is a hydroxyl, methoxy or acetoxy group;
R³ is hydrogen or a halogen atom
R⁴ is hydrogen or an acetoxy group; and
R⁵ is hydrogen or a methyl group,
with the proviso that at least one of R⁴ and R⁵ is hydrogen, or a pharmaceutically acceptable salt thereof.

16. The method of claim 15, wherein the halogen atom(s) is selected from the group consisting of chlorine, bromine and iodine.

17. The method of claim 13, wherein the compound is selected from the group consisting of 8-acetoxy-5-iodo-6-methoxypyrido[3,4-b]indole, 5,7-dibromo-6-hydroxypyrido [3,4-b]indole, 5,7-dibromo-6-acetoxypyrido [3,4-b]indole, 5,7-dibromo-6-acetoxy-9-methylpyrido [3,4-b]indole, 5,7-dibromo-6-hydroxy-9-methylpyrido[3,4-b]indole, 5,7-dichloro-6-hydroxypyrido[3,4-b]indole, 5,7-dichloro-6-hydroxy-9-methylpyrido [3,4-b]indole, 5,7-diiodo-6-hydroxypyrido[3,4-b]indole, and 5,7-diiodo-6-hydroxy-9-methylpyrido [3,4-b]indole.

18. The method of claim 13, wherein the compound is in the form of a hydrochloride salt.

19. A method of using a compound that elicits periodic amplified Ca²⁺ release in β-cells comprising obtaining a compound identified by the method of claim 2, preparing said compound as a pharmaceutical, and using said compound to treat defective insulin secretion related disorders.

20. The method of claim 19 wherein the defective insulin secretion related disorder is type 2 diabetes.
